(19) United States
(12) Patent Application Publication
(10) Pub. No.: US 2024/0185945 A1
(43) Pub. Date: Jun. 6, 2024

(54) SYSTEMS AND METHODS FOR MODELING AND DISPLAYING SWEETENER SYNERGY

(71) Applicant: WM. WRIGLEY JR. COMPANY, Chicago, IL (US)

(72) Inventors: Chia-Hua April HSU, Chicago, IL (US); Xiaoqun MO, Oak Park, IL (US)

(21) Appl. No.: 17/989,285

(22) Filed: Nov. 17, 2022

(51) Int. Cl. G16B 5/00; G16B 40/20; G16C 20/30; G16C 20/70

(52) U.S. Cl. CPC G16B 5/00; G16B 40/20; G16C 20/30; G16C 20/70

(57) ABSTRACT

Systems and computer-implemented methods of generating a model and using the model for determining synergy between a plurality of compounds in a mixture for applying to a human taste receptor are disclosed herein. The plurality of compounds in the mixture may be sweet-tasting compounds. The model may be generated based on a function, where the sigmoid function is approximated to a set of experimental data, and where the experimental data maps a combination of concentrations of compounds to a measured response of the taste receptor. Once generated, the model may then be used to determine synergy among the plurality of compounds.

### TECHNICAL FIELD

Various embodiments of the present disclosure relate generally to methods of generating models of edible compound mixtures that activate a taste receptor, and more particularly to methods of generating models of sweet-tasting compound mixtures, use of the models in analyzing synergy, and methods of preparing edible products using synergistic compound mixtures.

### BACKGROUND

Synergistic edible compound mixtures are often used to impart various properties to edible products. Such mixtures have been found to elicit an elevated response of a taste receptor. Examples of these mixtures include synergistic blends of sweeteners, which have been used for purposes such as enhancing sweetness of food and beverages, masking bitter or off tastes, and lowering production costs. Thus, understanding how a variety of sweet-tasting compounds interact in a mixture at the taste receptor level is desirable. Existing methods of studying interaction between compounds (e.g., sweeteners) in a mixture and their effect on a taste receptor often rely on the use of data which has been physically measured and may include varying the concentrations of each compound. However, these techniques may present challenges in determining the taste receptor response and synergy for data points that have not been physically measured.

Therefore, a need exists for a predictive model which may be used in determining taste receptor response and synergy for a wide range of edible compound mixtures, beyond the particular experimental outcomes in which a taste receptor response is physically measured.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to aspects of the present disclosure, systems and computer-implemented methods of generating a model and using the model for determining synergy between a plurality of compounds in a mixture for applying to a human taste receptor are disclosed herein. The systems and methods disclosed herein may also be used to prepare a synergistic sweetener composition based on the model.

In one aspect, an exemplary computer-implemented method may be executed by a processor and may include receiving a first data set comprising a plurality of data points, each data point comprising a concentration level of each compound in a mixture of a plurality of compounds. The method may also include receiving a second data set comprising a response level of a human taste receptor determined for each data point from the plurality of data points.

The method may further include generating a model of response levels of the human taste receptor based on the first data set and the second data set. The model of response levels may be generated by determining a mapping between each data point from the plurality of data points and the response level of the human taste receptor determined for each data point from the plurality of data points. The method may also include determining a first function of the concentration level of each compound in the mixture, the function having one or more unknown coefficients. Further, the method may include determining a second function of the concentration level of each compound in the mixture, the second function having the same form as the first function, by determining the one or more unknown coefficients by fitting the first function to the mapping between each data point from the plurality of data points and the response level of the human taste receptor determined for each data point from the plurality of data points.

In some embodiments, the first function is a sigmoid function. In some embodiments, the sigmoid function is based on a logistic function, a trigonometric function, or a Hill equation.

In some embodiments of the preceding aspects, which may be combined with any of the preceding embodiments, data associated with the model to a user are displayed on a user device.

In some embodiments of the preceding aspects, which may be combined with any of the preceding embodiments, the human taste receptor is a sweet taste receptor T1R2/T1R3 and each compound in the mixture is a sweet-tasting compound.

In some embodiments of the preceding aspects, which may be combined with any of the preceding embodiments, each compound in the mixture is selected from the group consisting of sugars, mogrosides, sweet amino acids, polyols, artificial sweeteners, natural sweeteners, and sweet tasting proteins.

In some embodiments of the preceding aspects, which may be combined with any of the preceding embodiments, the mixture comprises a first sweet-tasting compound, a second sweet-tasting compound, and a third sweet-tasting compound.

In some embodiments of the preceding aspects, which may be combined with any of the preceding embodiments, each of the first sweet-tasting compound, the second sweet-tasting compound, and the third sweet-tasting compound is selected from aspartame, sucrose, sucralose, rebaudioside A, rebaudioside D, rebaudioside M, thaumatin, neohespheridin, and S819 [1-((1H-pyrrol -2-yl)m ethyl)-3-(4-isopropoxyphenyl)thiourea].

In another aspect, a second exemplary method may be executed by a processor and may include receiving a plurality of data points associated with a plurality of sweeteners, each data point indicating a concentration level of a corresponding sweetener among the plurality of sweeteners. The method may also include determining, using a sweet taste receptor response model, synergy among the plurality of sweeteners based on the received plurality of data points, wherein the synergy is measured by an increase in a response level of a sweet taste receptor to the plurality of sweeteners. The method further includes prompting, via a user interface, a user to prepare a synergistic sweetener composition including the plurality of sweeteners in the respective concentration levels indicated in the plurailty of data points, such that the synergistic sweetener composition produces the synergy determined using the sweet taste receptor response model.

In some embodiments of the preceding aspects, which may be combined with any of the preceding embodiments, the increase in the response level of the sweet taste receptor is equal to or greater than 25%.

In some embodiments of the preceding aspects, which may be combined with any of the preceding embodiments, the plurality of sweeteners comprise at least two sweeteners.

In some embodiments of the preceding aspects, which may be combined with any of the preceding embodiments, the plurality of sweeteners comprise at least three sweeteners.

In some embodiments of the preceding aspects, which may be combined with any of the preceding embodiments, the response level of the sweet taste receptor is displayed by the processor and via the user interface.

In some embodiments of the preceding aspects, which may be combined with any of the preceding embodiments, the response level of the sweet taste receptor is displayed as luminosity.

In some embodiments of the preceding aspects, which may be combined with any of the preceding embodiments, the response level of the sweet taste receptor is displayed in a graph.

In some embodiments of the preceding aspects, which may be combined with any of the preceding embodiments, the sweet taste receptor response model is trained by determining, by the processor, a mapping between sample data points of sample sweeteners and corresponding response levels of the sweet taste receptor, each sample data point including a concentration level of a corresponding sample sweetener; determining, by the processor, a first function of the concentration levels of the sample sweeteners, the function having one or more unknown coefficients; and determining, by the processor, a second function of the concentration levels of the sample sweeteners, the second function having the same form as the first function, by determining the one or more unknown coefficients by fitting the first function to the mapping between the sample data points and the corresponding response levels of the sweet taste receptor.

In yet another aspect of the present disclosure, a third exemplary method includes preparing a synergistic sweetener composition with two or more sweeteners in respective concentrations recommended by a sweet taste receptor model. The model may be generated based on a plurality of data points associated with sweetener mixtures, each data point comprising a concentration level of each sweetener in each sweetener mixture and sweet taste receptor data comprising each response level of a sweet taste receptor T1R2/T1R3 determined for each data point from the plurality of data points. The model may be analyzed to determine synergy among sweeteners in sweetener mixtures and respective concentration levels of the sweeteners at which synergy is demonstrated in order to provide a recommendation for a synergistic composition.

In some aspects, provided herein is a sweetener composition prepared according to any one of the embodiments of the preceding methods.

In some aspects, provided herein is a confectionery product comprising any of the sweetener compositions described herein. In some embodiments, the confectionery product is selected from a group consisting of hard candy, soft candy, mints, chewing gum, gelatins, chocolate, fudge, jellybeans, fondant, licorice, hard-panned candy, toffee, and taffy.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a flow chart illustrating an exemplary method of generating a model, according to one or more embodiments.

FIG. 2 depicts an exemplary method for preparing a synergistic sweetener composition using a model, according to one or more embodiments.

FIG. 3 depicts a flowchart illustrating an exemplary method of training a model, according to one or more embodiments.

FIG. 4 illustrates an implementation of a computer system that may execute techniques presented herein.

FIGS. 5A-5C depict graphs that compare experimental results of applying varying concentrations of two sweeteners to the sweet taste receptor with results generated by a model, as discussed in Example 1.

FIG. 6 depicts graphs that compare experimental results of applying varying concentrations of three sweeteners to the sweet taste receptor with results generated by a model, as discussed in Example 2.

FIGS. 7A-7C each depict a graph displaying synergy in a mixture of two sweeteners as determined using a model, as discussed in Example 3.

FIG. 8 depicts a graph displaying synergy in a mixture of three sweeteners determined using a model, as discussed in Example 4.

FIG. 9A depicts graphs displaying synergy between three different mixtures of two sweeteners, as discussed in Example 6.

FIG. 9B depicts a graph generated by reconstructing the data from the graphs shown in FIG. 9A to display synergy between three sweeteners using a model, as discussed in Example 6.

### DETAILED DESCRIPTION

Various embodiments of the present disclosure relate generally to methods of generating models of edible compound mixtures that activate a taste receptor. More particularly, various embodiments of the present disclosure relate to computer-implemented methods of generating models of sweet-tasting compound mixtures which determine sweet taste receptor T1R2/T1R3 response, use of the models in analyzing synergy between the sweet-tasting compound mixtures, and methods of preparing edible products using synergistic compound mixtures determined using the models.

As discussed above, certain edible compounds (e.g., sweeteners) may be combined in a mixture in concentrations (i.e., concentration levels) that have a synergistic effect on a taste receptor. A synergistic response of the taste receptor may be determined by varying the concentrations of the compounds in the mixture. In certain scenarios, there may be a desire to determine a synergistic effect between a combination of three different compounds on a taste receptor, when synergy has already been determined between two out of the three compounds. However, this may require an extensive amount of testing and access to large amounts of data that may be unavailable. Furthermore, linear interpolation may be considered sub-optimal in predicting the response of a taste receptor to arbitrary concentrations of three or more compounds.

Therefore, the embodiments of the present disclosure are directed to solving, mitigating, or rectifying the above-mentioned issues by generating a model that can be used to forecast the expected response of a taste receptor, such as the sweet taste receptor T1R2/T1R3, to varying concentrations of compounds from a plurality of compounds in a mixture and capture synergies among the compounds. The model of the present disclosure may provide for the interpolation and extrapolation of the expected response of the human sweet taste receptor T1R2/T1R3 beyond and between the specific data points that are physically measured. The model may provide for a variety of uses, including using the model to predict taste receptor response of many different compound mixtures and to determine synergies thereof.

The present disclosure provides systems and methods for receiving data related to compounds in a compound mixture for applying to a human taste receptor and developing a model based on the data. The data may include the concentrations of each compound and the corresponding response level of the human taste receptor. The model may be generated by selecting a functional type for the model, and generating a closed-form equation that has multiple variables, each variable corresponding to a concentration of a compound of a mixture that is applied to a taste receptor.

In some examples herein, the computer-based model may be used for determining sweet taste receptor T1R2/T1R3 response based on varying concentrations of at least two sweet-tasting compounds. The model may be derived from a functional assay of the sweet taste receptor, where the response of the T1R2/T1R3 sweet taste receptor is measured in response to varying concentrations of the at least two sweet-tasting compounds. The functional assay produces a plurality of data points. In some examples, the data points correspond to a concentration of a first sweet-tasting compound, a concentration of a second sweet-tasting compound, and a concentration of a third sweet-tasting compound. Further, the data used to derive the model of the present disclosure may include an indication of a level of response of the sweet taste receptor when these concentrations are applied thereto.

Once generated, the model may be used to determine synergy among compounds in a mixture. Synergy may be determined when a response level of the taste receptor to the mixture of compounds is higher than a sum of the response levels of the taste receptor to each individual compound. In some embodiments, the computer-based model may be used in preparing a synergistic sweetener composition. Such sweetener compositions may be used to enhance the sweet taste of food products, including confectionery products.

Although the models described herein are directed to the activation of the human sweet taste receptor with sweeteners, the models of the present disclosure are applicable to other taste receptors, for example, umami taste receptors, that can be activated by one, two, three or more receptor activating compounds.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.” As used herein, the singular forms “a,” “an,” and “the” include plural reference unless the context dictates otherwise. Relative terms such as “about,” “substantially,” and “approximately” refer to being nearly the same as a referenced number or value, and should be understood to encompass a variation of ±5% of a specified amount or value.

As used herein, “taste” refers to a sensation caused by activation or inhibition of receptor cells in a subject's oral cavity. Different types of taste may include sweet, sour, salt, bitter, kokumi, umami, and any combination thereof.

The terms “sweetener” and “sweet tasting compound” may refer to a ligand or a compound that is capable of binding to T1R2/T1R3 and imparting or enhancing a sweet taste. For example, “sweet tasting compounds,” may include, without limitation, sugars, mogrosides, polyols, D-amino acids, sweet proteins, artificial sweeteners, sulfamates, and steviol glycosides.

As used herein, “synergy,” or “synergistic response” may refer to an effect produced by two or more individual components in which the total effect produced by these components, when utilized in combination, is greater than the sum of the individual effects of each component acting alone.

As used herein, a “model” or “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. Any suitable type of training may be used, e.g., stochastic, gradient boosted, random seeded, recursive, epoch or batch-based, etc.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

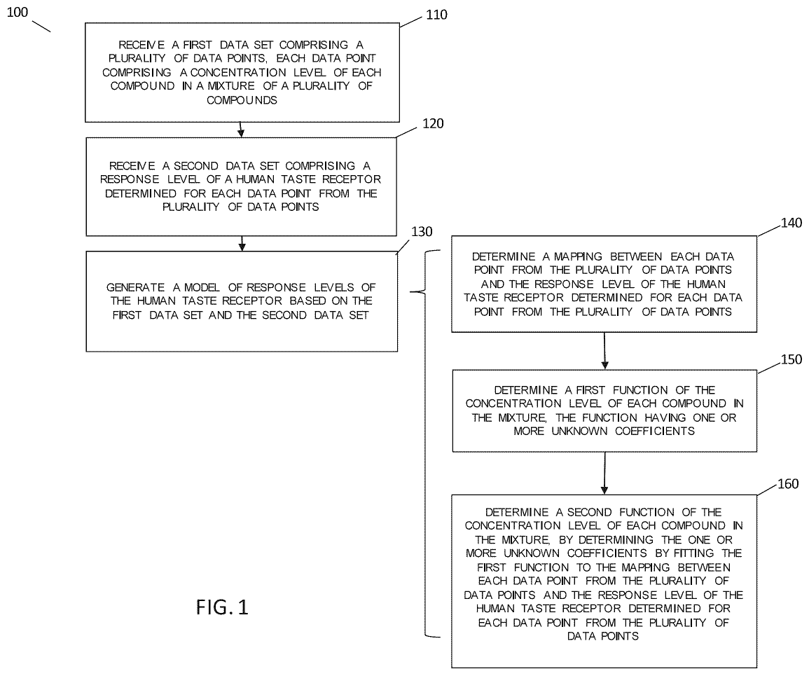

Referring now to the appended drawings, FIG. 1 depicts a flowchart of an exemplary method 100 of generating a model according to the present disclosure. The model may be configured to provide data for determining synergy between a plurality of compounds in a mixture for applying to a human taste receptor, such as the sweet taste receptor T1R2/T1R3. According to embodiments, the model is capable of being implemented in computer software on a desktop, laptop, notebook, handheld, or server-class computing device. Further, method 100 may be implemented in computer software and is capable of being executed on similar computing devices.

Method 100 begins at step 110. In step 110, a first data set is received by a processor. The first data set includes a plurality of data points. Each data point includes a concentration level of each compound in a mixture of a plurality of compounds. In some examples, the first data set is received from a database or a file. The plurality of data points may be represented as a data structure, such as an array, matrix, vector, lookup table or combinations thereof, that can be stored in the memory of a computer, or on a peripheral or network-attached storage device.

In step 120, a second data set is received by the processor. The second data set includes a response level of a human taste receptor determined for each data point from the plurality of data points received in step 110. The first data set and the second data set may be based on experimental data obtained from a functional assay of the human taste receptor, where the response of the receptor is measured in response to varying concentrations of each compound in the mixture of the plurality of compounds. In some examples, the first data set and the second data set may be received together as a single data set. The data points may define a particular response of the receptor when certain concentrations of different compounds are applied thereto. For example, each data point may be represented as an n-tuple, in which the first n-1 elements represent concentrations of different compounds that have been applied to the sweet taste receptor T1R2/T1R3. The nth element then represents the response level of the receptor to the given concentrations. This response level can be represented visually as a level of luminosity.

The database or file storing the first data set and the second data of experimental data may be populated using various data population methods. For example, an end user (such as an experimenter) may access a computer interface (such as a graphical user interface (GUI) or command-line interface (CLI)) and manually enter the data points (corresponding to the concentrations of compounds) and the corresponding measured luminescence of the receptors. Further, in other embodiments, the data points and luminescence may be transmitted as a file or data stream to a receiving process on a computer, where the receiving process automatically populates the database. In still other embodiments, an apparatus configured to conduct the measurement of luminescence of the sweet taste receptor based on detected concentrations of compounds is further configured to automatically transmit the concentrations and luminescence data to a receiving process for subsequent entry into the database or file. Moreover, a process that measures the luminescence of the sweet taste receptor may itself be configured to directly access and populate the database or file.

In step 130, a model of the response level of the human taste receptor (e.g., sweet taste receptor) is generated based on the first data set and the second set. Steps 140-160 are performed to generate the model after the data is received in preceding steps.

The experimental data received from the functional assay is processed in step 140. Step 140 includes determining a mapping between each data point from the plurality of data points and the response level of the human taste receptor determined for each data point from the plurality of data points. In step 150, a first function of the concentration level of each compound in the mixture is determined. The function may have one or more unknown coefficients. In aspects of the present disclosure, determining a first function may include selecting a sigmoid function from a plurality of sigmoid functions. For example, a sigmoid function may be selected from among a plurality of sigmoid functions to approximate the experimental data. A functional type for modeling receptor response is one with a sigmoidal (or “S”) shape, and many biological processes can be modeled using this shape. Several sigmoid functions are used in biological modeling. Among the functions used are the logistic function, the Gompetz sigmoid function, trigonometric functions, and the Hill equation.

The logistic function can be represented as:

$$f ( x ) = \frac{1}{1 + {e}^{- x}} .$$

The Gompetz sigmoid function can be represented as f(x)=ae^{be}^{^{−cx}}, where a, b, and c may be varied to change, respectively, asymptote, displacement, and growth rate. A trigonometric function that evinces a sigmoid shape is the hyperbolic tangent function, or tanh (x). Finally, the Hill equation can be represented as

$$f ( x ) = \frac{{x}^{n}}{1 + {x}^{n}} .$$

Further, other sigmoid functions can be obtained using linear combinations of other known sigmoid functions, as well as by multiplication and/or superposition of known sigmoid functions.

Thus, according to embodiments, any one of the aforementioned functions (or, indeed, another sigmoid function) may be selected in step 150. Such functions may be stored, for example, as equations in a database, where parameter values (e.g., the value of “n” in the Hill equation) are able to be varied.

In step 160, a second function of the concentration level of each compound in the mixture is determined. The second function has the same form as the first function. In order to perform step 160, an expression representing a combination of compound concentrations is substituted as the independent variable of the selected sigmoid function. In one or more embodiments, an expression for the concentrations of the compounds is defined as the sum of the products of the compound concentrations and the binding constants, as shown by the following expression:

aₓx+a_{y}y+a_{z}z+a_{xy}xy+a_{xz}xz+a_{yz}yz+a_{xyz}xyz

In this expression, x, y, and z are the concentrations of each compound (e.g., first sweet-tasting compound, second sweet-tasting compound, and third sweet-tasting compound respectively), and the an coefficients are unknown binding constants. Thus, assuming that the selected sigmoid function is the Hill equation, after substitution of the above expression, the sigmoid function can be represented as:

$$f ( x , y , z ) = \frac{{( {a}_{x} x + {a}_{y} y + {a}_{z} z + {a}_{xy} xy + {a}_{xz} xz + {a}_{yz} yz + {a}_{xyz} xyz )}^{n}}{1 + {( {a}_{x} x + {a}_{y} y + {a}_{z} z + {a}_{xy} xy + {a}_{xz} xz + {a}_{yz} yz + {a}_{xyz} xyz )}^{n}}$$

Similarly, if the selected sigmoid function is the logistic function, then the sigmoid function can be represented as:

$$f ( x , y , z ) = \frac{1}{1 + {e}^{- ( {a}_{x} x + {a}_{y} y + {a}_{z} z + {a}_{xy} xy + {a}_{xz} xz + {a}_{yz} yz + {a}_{xzy} xzy )}}$$

Once the expression for the compound concentrations is substituted into the selected sigmoid function, values for the unknown coefficients in the sigmoid function are determined. The one or more unknown coefficients are determined by fitting the first function (e.g., sigmoid function) to the mapping between each data point from the plurality of data points and the response level of the human taste receptor determined for each data point from the plurality of data points. For example, in the case of the Hill equation, the unknown coefficients are the binding constants aₓ, a_{y}, a_{z}, a_{xz}, a_{yz}, and a_{xyz}, as well as the exponent n. Similarly, for the logistic function, the unknown coefficients are the aforementioned binding constants. For the Gompetz sigmoid function, the unknown coefficients are the binding constants, as well as asymptote, displacement, and growth rate.

According to embodiments, the unknown coefficients of the updated sigmoid function generated at step 160 are determined using nonlinear regression. That is, the coefficients of the function f(x, y, z) are selected to better approximate (or “fit”) the experimental data that was received in steps 110 and 120. In this manner, the difference between the simulated luminosity values generated by the sigmoid function and the actual luminosity levels specified in the experimental data is reduced.

In at least one example, method 100 may include a further step of determining whether any sigmoid functions remain to be evaluated to approximate the experimental data. For example, assuming that the first selected sigmoid function was the Hill equation, and that the method is adapted to further evaluate sigmoid functions having the hyperbolic tangent, logistic, or Gompetz forms, then method 100 may determine that these additional sigmoid functions remain to be evaluated. If there are more sigmoid functions to be evaluated, then method 100 proceeds back to step 150, where a next sigmoid function is selected. The method may then proceed to step 160 for the next selected sigmoid function. In examples wherein multiple sigmoid functions are evaluated, the sigmoid function having the lowest approximation error among all of the evaluated sigmoid functions is selected as the model.

After the model has been generated in method 100, data associated with the model may be displayed to a user on a user device. For example, the data associated with the model may be displayed as a luminosity plot. The luminosity plot may show the human taste receptor response (luminosity level) to compounds in a mixture of compounds. In aspect of the present disclosure, sweeteners or sweet-tasting compounds may be used as compounds in a mixture of a plurality of compounds that the experimental data used to generate the model is based on. For example, prior to the steps of receiving data in method 100, a functional assay of the sweet taste receptor T1R2/T1R3 may be performed where the response of the receptor is measured in response to varying concentrations of two or more sweet-tasting compounds.

The sweet-tasting compounds that may be used in embodiments of the present disclosure may include sugars, sweet amino acids, polyols, artificial sweeteners, natural sweeteners, and sweet tasting proteins. Exemplary sweet-tasting compounds may include, but are not limited to, sucrose, fructose, glucose, high fructose corn syrup, tagatose, galactose, ribose, xylose, arabinose, rhamnose, erythritol, xylitol, mannitol, sorbitol, inositol, saccharine, methyl chavicol, Theasaponin El, Advantame, Acesulfame K, Alitame, Aspartame, CH 401, Dulcin, Neotame, Cyclamate, Sucralose, Superaspartame, Cynarin, Glycyphyllin, Rebaudioside C, Abrusoside A, Abrusoside B, Abrusoside C, Abrusoside D, Abrusoside E, Apioglycyrrhizin, Araboglycyrrhizin, Baiyunoside, Brazzein, Bryodulcoside, Carnosifloside V, Carnosifloside VI, D. cumminsii, Cyclocarioside A, Cyclocarioside I, Dulcoside A, Glycyrrhizic Acid, Hernandulcin, 4beta-hydroxy-Hesperitin-7-Glucoside Dihydrochalcone, Huangqioside E, Huangqioside E, 3-Hydroxyphloridzin, 2,3-Dihydro-6-Methoxy 3-O-Acetate, Mabinlin Maltosyl-Alpha-(1,6)-Neohesperidin Dihydrochalcone, Miracullin, Mogroside IIE, Mogroside III, Mogroside IIIE, Mogroside IV, Mogroside V, 11-Oxo Mogroside V, Monatin, Monellin, Monoammonium Glycyrrhizinate (Mag), Mukurozioside Iib, Naringin Dihydrochalcone, Neohesperdin (NHDC), Neohesperidin Dihydrochalcone (NHDHC), Neomogroside, Osladin, Pentadin, Perillartine, Periandrin I, Periandrin II, Periandrin III, Periandrin IV, Periandrin V, Phlomisoside I, Phlorizin, Phyllodulcin, Polypodoside A, Potassium magnesium calcium glycyrrhizin, Pterocaryosides A, Pterocaryosides B, Rebaudioside A, Rebaudioside B, Rebaudioside D, Rebaudioside M, Rubusoside, Scandenoside R6, SE-1 comprising the following structure (Formula I):

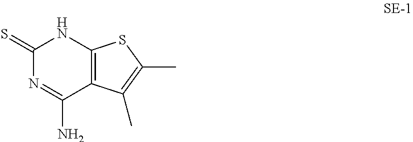

SE-2 comprising the following structure (Formula II):

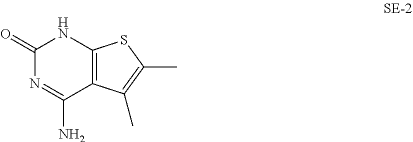

SE-3 comprising the following structure (Formula III):

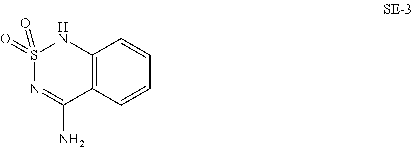 [SMILES: NC1=NS(=O)(=O)Nc2ccccc21]

SE-4 comprising the following structure (Formula IV):

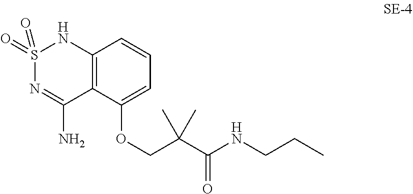 [SMILES: CCCNC(=O)C(C)(C)COc1cccc2c1C(N)=NS(=O)(=O)N2]

Siamenoside I, Sodium glycyrrhizinate, Steviolbioside, Stevioside, alpha-Glycosyl Suavioside A, Suavioside B, Suavioside G, Suavioside H, Suavioside I, Suavioside J, Sweelin, Thaumatin, Triammonium Glycyrrhizinate (TAG), Trilobatin, Curculin, Strogin 1, Strogin 2, Strogin 4, Miraculin, Hodulcin, Jujubasaponin II, Jujubasaponin III, Abrusoside E, Periandrinic acid I, monoglucuronide, Periandrinic acid II, monoglycuronide, Chlorogenic Acid, beta-(1,3-Hydroxy-4-methoxybenzyl)-Hespertin Dihydrochalcone, 3′-Carboxy-Hespertin Dihydrochalcone, 3′-Stevioside analogue, and S819[1-((1H-pyrrol-2-yl)methyl)-3-(4-isopropoxyphenyl)thiourea] comprising the following structure (Formula V):

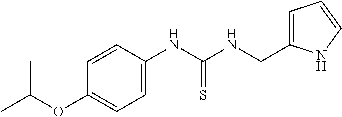 [SMILES: CC(C)Oc1ccc(NC(=S)NCc2ccc[nH]2)cc1]

In some examples, the model may be based on a first sweet-tasting compound and a second sweet-tasting compound selected from the group disclosed above. In other examples, the model may be based on a first sweet-tasting compound, a second sweet-tasting compound, and a third sweet-tasting compound may be selected from the group of sweet-tasting compounds disclosed above. In at least one example, a plurality of sweet-tasting compounds may include combinations of thaumatin, neohesperdin (NHDC), and aspartame. Other examples may include various combinations of aspartame, sucrose, sucralose, rebaudioside A, rebaudioside D, thaumatin, NHDC, and S819.

In some examples, the plurality of sweet-tasting compounds may include two or more compounds that bind to different sites of the T1R2/T1R3 sweet taste receptor. In at least one example, at least one compound in the mixture may bind to T1R2 and at least one compound in the mixture may bind to T1R3. For example, at least one compound may bind to a seven-helical transmembrane domain (7TM), a venus flytrap domain, or a cysteine-rich domain of T1R2 and at least one compound may bind to a seven-helical transmembrane domain (7TM), a venus flytrap domain, or a cysteine-rich domain of T1R3. In certain examples, each compound in the mixture may bind to a different domain of T1R2 or a different domain of T1R3. In other examples, each compound in the mixture may bind to a same domain of T1R2 or a same domain of T1R3.

Once a model has been computed based on experimental data according to method 100, the model may then be used in a variety of applications. For example, the model may be used to determine a predicted receptor response for a given concentration of compounds. Alternatively, the model may be used to determine an unknown concentration of one compound, given specified concentrations for the other compounds, as well as a specific desired receptor response. The model may also be used to determine a set of combinations of compounds that give rise to at least a threshold receptor response. Further, the model may be used to determine synergy between two or more different compounds (e.g., sweet-tasting compounds) in a mixture of the compounds.

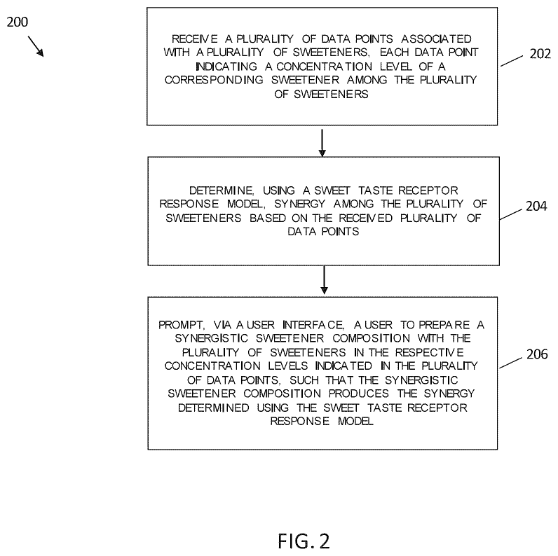

FIG. 2 depicts a flowchart of an exemplary method 200 of using a model according to the present disclosure. In particular, the method 200 may be performed in order to prepare a synergistic sweetener composition. Prior to preparing the synergistic sweetener composition, the method 200 details using a model to determine synergy among a plurality of sweeteners in a mixture of the sweeteners.

In step 202, a plurality of data points associated with a plurality of sweeteners is received. Each data point indicates a concentration level of a corresponding sweetener among a plurality of sweeteners. Each of the sweeteners in the plurality of sweeteners may be a sweetener from the group of sweet-tasting compounds disclosed above.

Next, in step 204, a sweet taste receptor response model is used to determine synergy among the plurality of sweeteners based on the received plurality of data points. For example, after a model that is derived from a functional assay of the human sweet taste receptor is generated according to method 100 as described above, the model may be used in step 204 to determine synergy among a plurality of sweeteners. In step 204, synergy is measured by an increase in a response level of the sweet taste receptor to the plurality of sweeteners. In one embodiment, the model may determine synergy between two or more compounds in a mixture on which the model is based when a response level of the human taste receptor to the mixture is higher than a sum of response levels of the human taste receptor to each individual compound.

As described above with respect to method 100, the response level of the human taste receptor (e.g., sweet taste receptor) may be displayed as luminosity in the form of a graph. For example, the processor may display the response level of the sweet taste receptor via a user interface. Using the model to determine synergy may include determining a synergy function obtained by subtracting a response level of the sweet taste receptor to each individual sweetener from the response level of the sweet taste receptor to the mixture of the two or more sweeteners. The synergy function used to determine synergy according to the present disclosure can be represented as:

Max(L(X&Y&Z)−L(X)−L(Y)−L(Z), 0)

In the above synergy function, L (luminescence) is the mathematically modeled normalized receptor response and X,Y, and Z are the three components (e.g., sweeteners) of the mixture. In some examples, synergy may be determined between sweeteners in a mixture of two sweeteners. In these examples, the synergy function may be represented as:

Max(L(X&Y)−L(X)−L(Y), 0)

Once the synergistic response is determined, the synergistic response may be plotted as a function of concentrations of each sweetener in the mixture. For example, the synergy function Syn(X,Y,Z) or Syn(X,Y) can be plotted vs. concentration of the sweeteners. The plotting may be displayed as graph on a user device via a user interface. The graph may display luminescence values ranging from 0 to 1. In some examples, synergy may be depicted as a (sweetness) signal increase. For example, the increase in the response level of the sweet taste receptor may be at least 25%. In some examples, the increase in the response level for the mixture may range from 25% to 60%.

After synergy is determined, method 200 proceeds to step 206. In step 206, a user is prompted via a user interface to prepare a synergistic sweetener composition with the plurality of sweeteners in the respective concentration levels indicated in the plurality of data points, such that the synergistic sweetener composition produces the synergy determined using the sweet taste receptor response model in step 204.

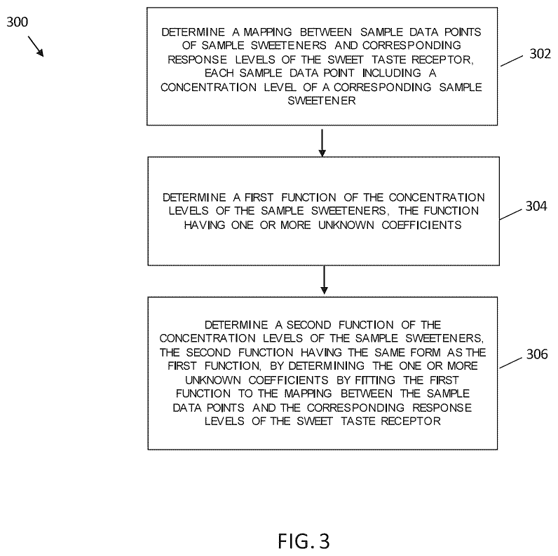

Before the model is used to determine synergy between multiple sweet-tasting compounds, the model is trained using a plurality of sample data points as shown in FIG. 3. FIG. 3 illustrates an exemplary method 300 of training the sweet taste receptor response model used in method 200.

In step 302, a mapping between sample data points of sample sweeteners and corresponding response levels of the sweet taste receptor is determined. Each sample data point includes a concentration level of a corresponding sample sweetener.

In step 304, a first function of the concentration levels of the sample sweeteners is determined. The function has one or more unknown coefficients. For example, the first function may be a sigmoid function as described above with respect to method 100.

In step 306, a second function of the concentration levels of the sample sweeteners is determined. The second function has the same form as the first function. The second function is determined by determining the one or more unknown coefficients by fitting the first function to the mapping between the sample data points and the corresponding response levels of the sweet taste receptor.

In some embodiments, the synergistic sweetener compositions produced according to the present disclosure may be used in any food or beverage composition. Such compositions may include, but are not limited to, baked goods, dairy products, carbonated and noncarbonated beverages, confectionery products, and the like. In particular, the synergistic sweetener compositions of the present disclosure may be used in confectionery products. The confectionery product may be in the form of hard candy, soft candy, mints, chewing gum, gelatins, chocolate, fudge, jellybeans, fondant, licorice, hard-panned candy, toffee, or taffy. The synergistic sweetener compositions may be used to enhance sweetness.

In addition to being configured to determine synergy among a plurality of compounds (e.g., sweeteners) in a mixture thereof, models of the present disclosure may also provide additional benefits. For example, the model may be capable of reconstructing a data set based on three compounds (3D) from experiments based on 1 compound (1D) or 2 compounds (2D), without having to conduct the experiment with the three compounds. For example, 2D experiments may be reconstructed from 1D diagonals, from 1D lines and/or from any scattered set of 2D points. In some embodiments, 3D data may be reconstructed from diagonals, from any 2D projection, from 1D lines and/or from any scattered set of 3D points.

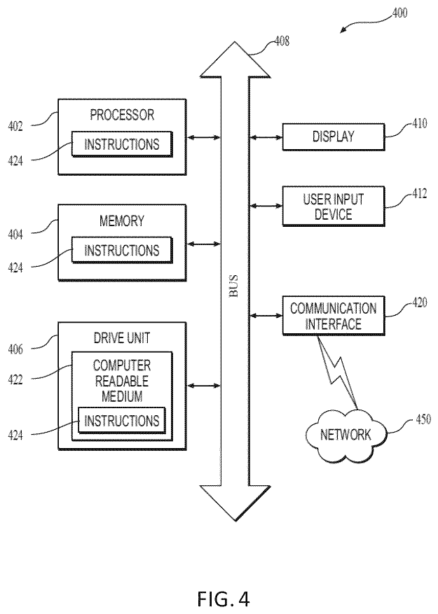

FIG. 4 illustrates an implementation of a computer system that may execute techniques presented herein. The computer system 400 can include a set of instructions that can be executed to cause the computer system 400 to perform any one or more of the methods or computer based functions disclosed herein. The computer system 400 may operate as a standalone device or may be connected, e.g., using a network, to other computer systems or peripheral devices.

In a networked deployment, the computer system 400 may operate in the capacity of a server or as a client user computer in a server-client user network environment, or as a peer computer system in a peer-to-peer (or distributed) network environment. The computer system 400 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 400 can be implemented using electronic devices that provide voice, video, or data communication. Further, while a single computer system 400 is illustrated, the term “system” shall also be taken to include any collection of systems or sub-systems that individually or jointly execute a set, or multiple sets, of instructions to perform one or more computer functions.

As illustrated in FIG. 4, the computer system 400 may include a processor 402, e.g., a central processing unit (CPU), a graphics processing unit (GPU), or both. The processor 402 may be a component in a variety of systems. For example, the processor 402 may be part of a standard personal computer or a workstation. The processor 402 may be one or more general processors, digital signal processors, application specific integrated circuits, field programmable gate arrays, servers, networks, digital circuits, analog circuits, combinations thereof, or other now known or later developed devices for analyzing and processing data. The processor 402 may implement a software program, such as code generated manually (i.e., programmed).

The computer system 400 may include a memory 404 that can communicate via a bus 408. The memory 404 may be a main memory, a static memory, or a dynamic memory. The memory 404 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 404 includes a cache or random-access memory for the processor 402. In alternative implementations, the memory 404 is separate from the processor 402, such as a cache memory of a processor, the system memory, or other memory. The memory 404 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts or tasks illustrated in the figures or described herein may be performed by the programmed processor 402 executing the instructions stored in the memory 404. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 400 may further include a display unit 410, such as a liquid crystal display (LCD), an organic light emitting diode (OLED), a flat panel display, a solid-state display, a cathode ray tube (CRT), a projector, a printer or other now known or later developed display device for outputting determined information. The display 410 may act as an interface for the user to see the functioning of the processor 402, or specifically as an interface with the software stored in the memory 404 or in the drive unit 406.

Additionally or alternatively, the computer system 400 may include an input device 412 configured to allow a user to interact with any of the components of system 400. The input device 412 may be a number pad, a keyboard, or a cursor control device, such as a mouse, or a joystick, touch screen display, remote control, or any other device operative to interact with the computer system 400.

The computer system 400 may also or alternatively include a disk or optical drive unit 406. The disk drive unit 406 may include a computer-readable medium 422 in which one or more sets of instructions 424, e.g. software, can be embedded. Further, the instructions 424 may embody one or more of the methods or logic as described herein. The instructions 424 may reside completely or partially within the memory 404 and/or within the processor 402 during execution by the computer system 400. The memory 404 and the processor 402 also may include computer-readable media as discussed above.

In some systems, a computer-readable medium 422 includes instructions 424 or receives and executes instructions 424 responsive to a propagated signal so that a device connected to a network 450 can communicate voice, video, audio, images, or any other data over the network 450. Further, the instructions 424 may be transmitted or received over the network 450 via a communication port or interface 420, and/or using a bus 408. The communication port or interface 420 may be a part of the processor 402 or may be a separate component. The communication port 420 may be created in software or may be a physical connection in hardware. The communication port 420 may be configured to connect with a network 450, external media, the display 410, or any other components in system 400, or combinations thereof. The connection with the network 450 may be a physical connection, such as a wired Ethernet connection or may be established wirelessly as discussed below. Likewise, the additional connections with other components of the system 400 may be physical connections or may be established wirelessly. The network 450 may alternatively be directly connected to the bus 408.

While the computer-readable medium 422 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 422 may be non-transitory, and may be tangible.

The computer-readable medium 422 can include a solid-state memory such as a memory card or other package that houses one or more non-volatile read-only memories. The computer-readable medium 422 can be a random-access memory or other volatile re-writable memory. Additionally or alternatively, the computer-readable medium 422 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 400 may be connected to one or more networks 450. The network 450 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 450 may include wide area networks (WAN), such as the Internet, local area networks (LAN), campus area networks, metropolitan area networks, a direct connection such as through a Universal Serial Bus (USB) port, or any other networks that may allow for data communication. The network 450 may be configured to couple one computing device to another computing device to enable communication of data between the devices. The network 450 may generally be enabled to employ any form of machine-readable media for communicating information from one device to another. The network 450 may include communication methods by which information may travel between computing devices. The network 450 may be divided into sub-networks. The sub-networks may allow access to all of the other components connected thereto or the sub-networks may restrict access between the components. The network 450 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

The following examples are intended to illustrate the present disclosure without, however, being limiting in nature. It is understood that the present disclosure encompasses additional embodiments consistent with the foregoing description and following examples.

### EXAMPLES

### Example 1: (2D) Modeling of Binary Combinations of NHDC, Thaumatin, and Aspartame Activation of the Human Sweet Taste Receptor

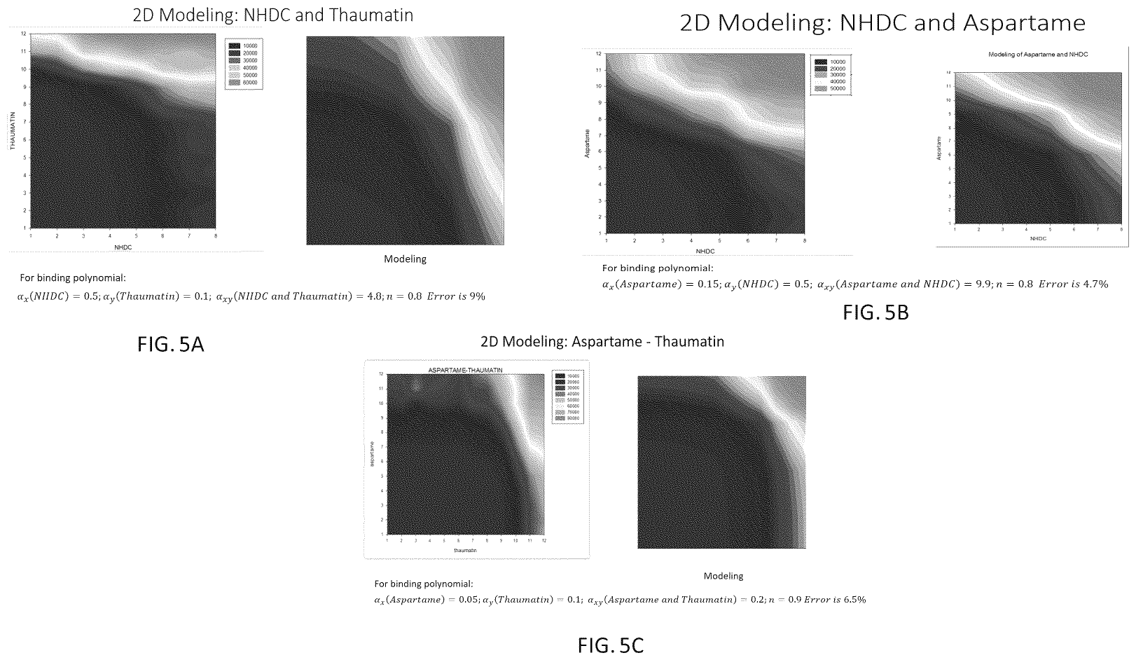

The present example describes the modeling of human sweet receptor activation by a combination of the NHDC and thaumatin shown in FIG. 5A, a combination of NHDC and aspartame shown in FIG. 5B, and a combination of aspartame and thaumatin shown in FIG. 5C.

Methods: In accordance with the techniques described above, the generation of a model for combining different concentrations of NHDC and thaumatin as shown in FIG. 5A, the generation of a model for combining different concentrations of NHDC and aspartame as shown in FIG. 5B, and the generation of aa model for combining different concentrations of aspartame and thaumatin as shown in FIG. 5C are described. For each embodiment, a predictive model based on the Hill equation was generated. The models were generated based on experimental data comprising 1,152 data points, representing 12 concentrations of aspartame, 8 concentrations of NHDC, and 12 concentrations of thaumatin. The concentration of aspartame was varied from 0 to 10 mM, the concentration of NHDC was varied from 0 to 1 mM, and the concentration of thaumatin was varied from 0 to 100 mM. With respect to the mixture of NHDC and thaumatin (FIG. 5A), the Hill equation that most closely approximated the experimental data had coefficients aₓ=0.5, a_{y}=0.1, a_{xy}=4.8, and n=0.8. With respect to the mixture of NHDC and aspartame (FIG. 5B), the Hill equation that most closely approximated the experimental data had coefficients aₓ=0.15, a_{y}=0.5, a_{xy}=9.9, and n=0.8. With respect to the mixture of aspartame and thaumatin (FIG. 5C), the Hill equation that most closely approximated the experimental data had coefficients aₓ=0.05, a_{y}=0.1, a_{xy}=0.2, and n=0.9.

Results: FIGS. 5A-5C are each a visual comparison of the modeling results of the sigmoid function (Hill equation) with experimental results of applying varying concentrations of two sweeteners to the sweet receptor of a human, where the same concentrations of sweeteners are input to the model. In FIG. 5A, thaumatin tick marks represent concentrations of 0, 0.06, 0.1, 0.3, 0.6, 1, 3, 6, 10, 30, 60, 100 (uM) and NHDC tick marks represent concentrations of 0, 0.01, 0.03, 0.06, 0.1, 0.3, 0.6, 1 (mM). In FIG. 5B, aspartame tick marks represent 0, 0.006, 0.01, 0.03, 0.06, 0.1, 0.3, 0.6, 1, 3, 6, 10 (mM) and NHDC tick marks represent concentrations of 0, 0.01, 0.03, 0.06, 0.1, 0.3, 0.6, 1 (mM). In FIG. 5C, thaumatin tick marks represent concentrations of 0, 0.06, 0.1, 0.3, 0.6, 1, 3, 6, 10, 30, 60, 100 (uM) and aspartame tick marks represent 0, 0.006, 0.01, 0.03, 0.06, 0.1, 0.3, 0.6, 1, 3, 6, 10 (mM).

### Example 2: (3D) Modelling of NHDC, Thaumatin, and Aspartame Activation of the Human Sweet Taste Receptor

The present example describes the modeling of human sweet receptor activation by a combination of the sweetener compounds NHDC, thaumatin, and aspartame.

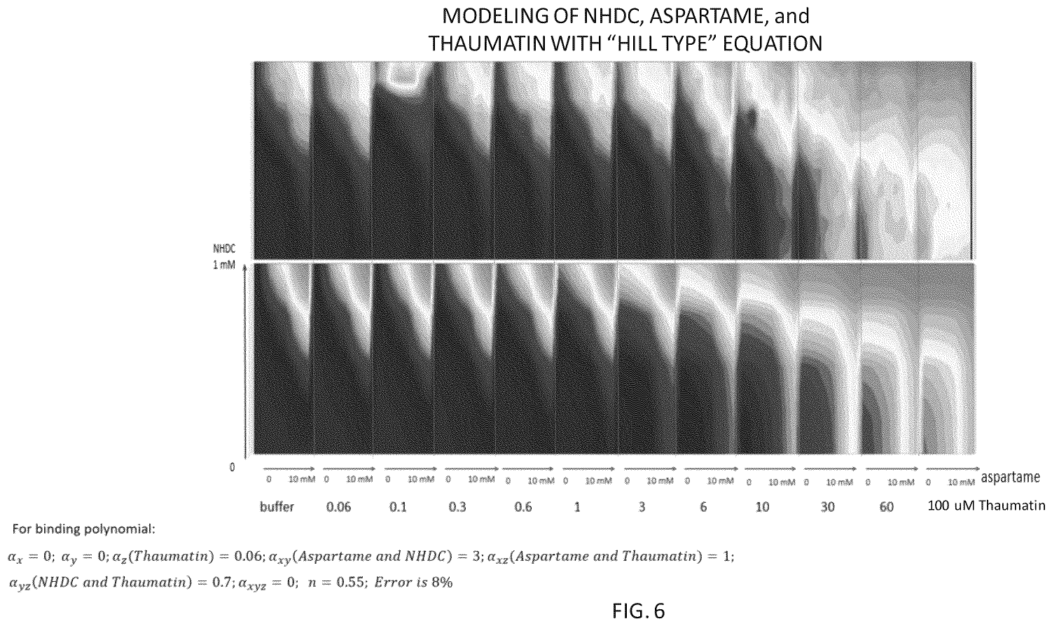

Methods: In accordance with the techniques described above, a Hill equation based model was generated to model combinations of aspartame, NHDC, and thaumatin. The experimental data comprises 1,152 data points, representing 12 concentrations of aspartame, 8 concentrations of NHDC, and 12 concentrations of thaumatin. In this example, the Hill equation most closely approximates the experimental data, as described below with reference to FIG. 6.

Results: FIG. 6 shows a comparison of experimental and model results for 12 concentrations of thaumatin, where, for each concentration of thaumatin, the concentration of NHDC was varied from 0 to 1 mM, while the concentration of aspartame was varied from 0 to 10 mM. In this case, the Hill equation approximates the experimental data with the lowest rate of error. The coefficients are as follows: aₓ=0, a_{y}=0, a_{z}=0.06, a_{xy}=3, a_{yz}=0.7, a_{xz}=1, a_{xyz}=0, and n=0.55, which gave rise to a sigmoid function represented by the equation

$$f ( x , y , z ) = \frac{{( 0.06 z + 0.7 yz + 3 xy + 1 xz )}^{0.55}}{1 + {( 0.06 z + 0.7 yz + 3 xy + 1 xz )}^{0.55}} .$$

### Example 3: (2D) Maps Compound Synergy of Binary Combinations of NHDC, Thaumatin, and Aspartame

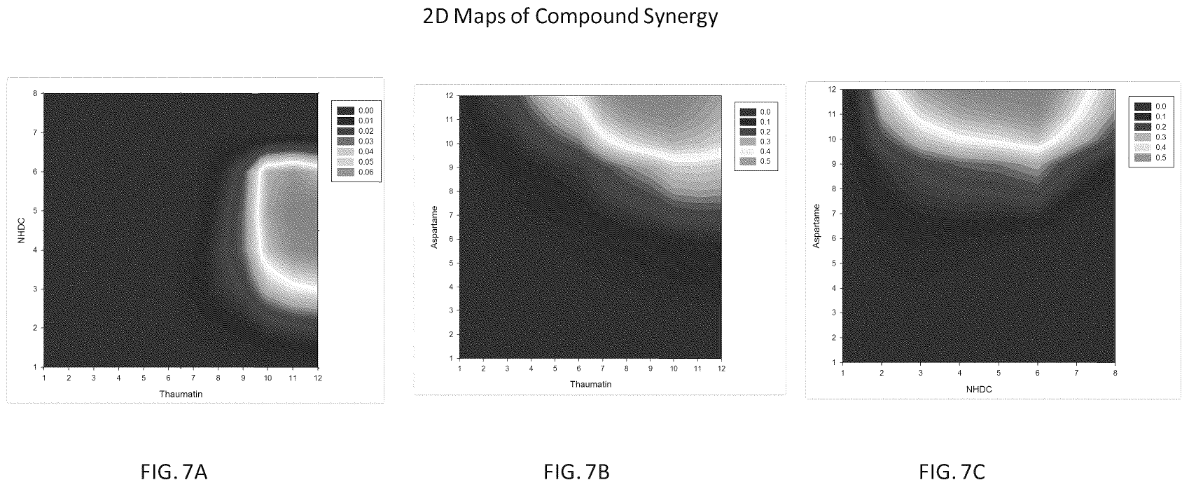

The present example describes plotting compound synergy between NHDC and thaumatin using a model generated in accordance with techniques described above as shown in FIG. 7A, plotting compound synergy between aspartame and thaumatin using a model generated in accordance with techniques described above as shown in FIG. 7B, and plotting compound synergy between aspartame and NHDC using a model generated in accordance with techniques described above as shown in FIG. 7C.

Each of FIGS. 7A-7C shows a plot of the fitted response of the mixture of two compounds—L(X&Y)—after subtracting the fitted individual responses for each component of the mixture, namely L(X) and L(Y), and L(Z) (X and Y are binary combinations of aspartame, NHDC and thaumatin). 2D synergy by subtraction is expressed by the following formula:

Max(L(X&Y)−L(X)−L(Y), 0).

As in previous figures described above, the difference is plotted as a function of aspartame, NHDC and Thaumatin concentrations. Aspartame tick marks represented as 2-12 represent concentrations of 0, 0.006, 0.01, 0.03, 0.06, 0.1, 0.3, 0.6, 1, 3, 6, 10 (mM), respectively. NHDC tick marks represented as 10-90 represent concentrations of 0, 0.01, 0.03, 0.06, 0.1, 0.3, 0.6, 1 (mM), respectively. Thaumatin tick marks represented as 2-12 represent concentrations of 0, 0.06, 0.1, 0.3, 0.6, 1, 3, 6, 10, 30, 60, 100 (uM). The Luminiscence(L) values range from 0 to 1.

Results: FIG. 7A shows that there is very little synergy between NHDC and thaumatin. FIGS. 7B and 7C shows high synergy between aspartame and thaumatin as well as aspartame and NHDC, respectively.

### Example 4: (3D) Maps Compound Synergy of NHDC, Thaumatin, and Aspartame Mixture

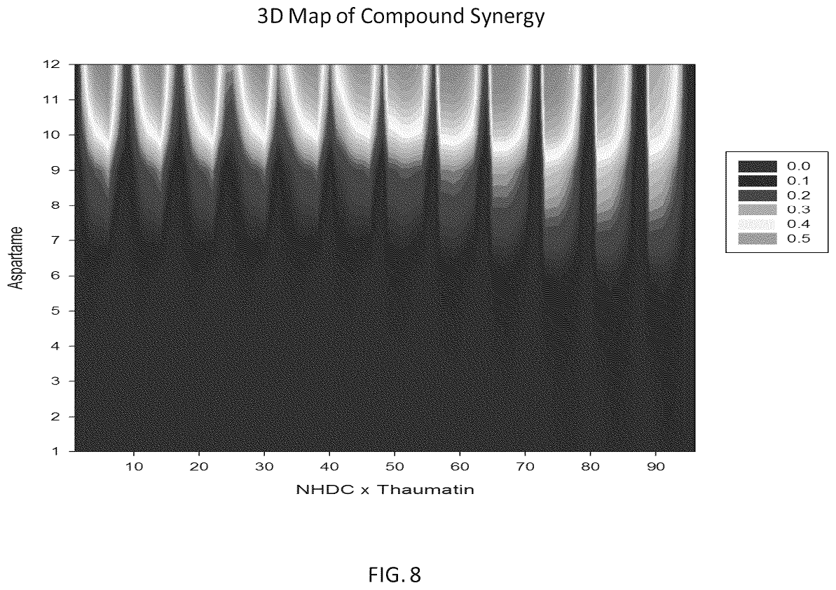

The present example describes plotting compound synergy between NHDC, thaumatin, and aspartame using a model generated in accordance with techniques described above as shown in FIG. 8.

FIG. 8 shows a plot of the fitted response of the mixture of three compounds—L(X&Y&Z)—after subtracting the fitted individual responses for each component of the mixture, namely L(X) and L(Y), and L(Z) (X is aspartame, Y is NHDC, and Z is thaumatin). 3D synergy by subtraction is expressed by the following formula:

ti Max(L(X&Y&Z−L(X)−L(Y)−L(Z) , 0).

As in previous figures described above, the difference is plotted as a function of aspartame and NHDC concentrations, in panels representing fixed amounts of thaumatin. Aspartame tick marks represented as 2-12 represent concentrations of 0, 0.006, 0.01, 0.03, 0.06, 0.1, 0.3, 0.6, 1, 3, 6, 10 (mM), respectively. NHDC tick marks represented as 10-90 represent concentrations of 0, 0.01, 0.03, 0.06, 0.1, 0.3, 0.6, 1 (mM), respectively. Thaumatin panels 0-90 represent concentrations of 0, 0.06, 0.1, 0.3, 0.6, 1, 3, 6, 10, 30, 60, 100 (uM). The Luminiscence(L) values range from 0 to 1.

Results: FIG. 8 shows that the synergy reaches values of 0.5.

### Example 5: Synergy Effects Observed in Various Combinations of Sweeteners

The present example describes sweet taste receptor signal increases calculated for the following sweeteners: aspartame, sucrose, sucralose, rebaudioside A, rebaudioside D, thaumatin, NHDC, and S819 are present in various combinations in a binary mixture as shown in Table 1.

TABLE 1
Synergy effects observed in binary compound mixtures

| | Aspar- | | | | | |
|---|---|---|---|---|---|---|
| | tame | sucrose | sucralose | Reb A | Thaumatin | NHDC |
| Reb A | | 0.5 | 0.3 | | | 0.25 |
| Reb D | | 0.25 | 0.3 | | | |
| Thaumatin | 0.5 | | | 0.4 | | |
| NHDC | 0.5 | | | | 0.06 | |
| S819 | | | | 0.25 | 0.4 | 0.4 |

Synergy between binary mixtures of the sweeteners was calculated as following: L(X&Y)/(L(X)+L(Y)). When both compounds are present in the binary mixtures as shown in Table 1, the increase in sweetness signal ranges from 25% to 50%.

Example 6: 3D Reconstruction From 2D Projections of NHDC, Rebaudioside A, and S819 Activation of the Human Sweet Taste Receptor

The present example describes the modeling of human sweet taste receptor activation by a combination of the NHDC, REB A, and S819 based on initial data consisting of three XY, XZ, and YZ planes, where Xis NHDC, Y is REB A, and Z is S819.

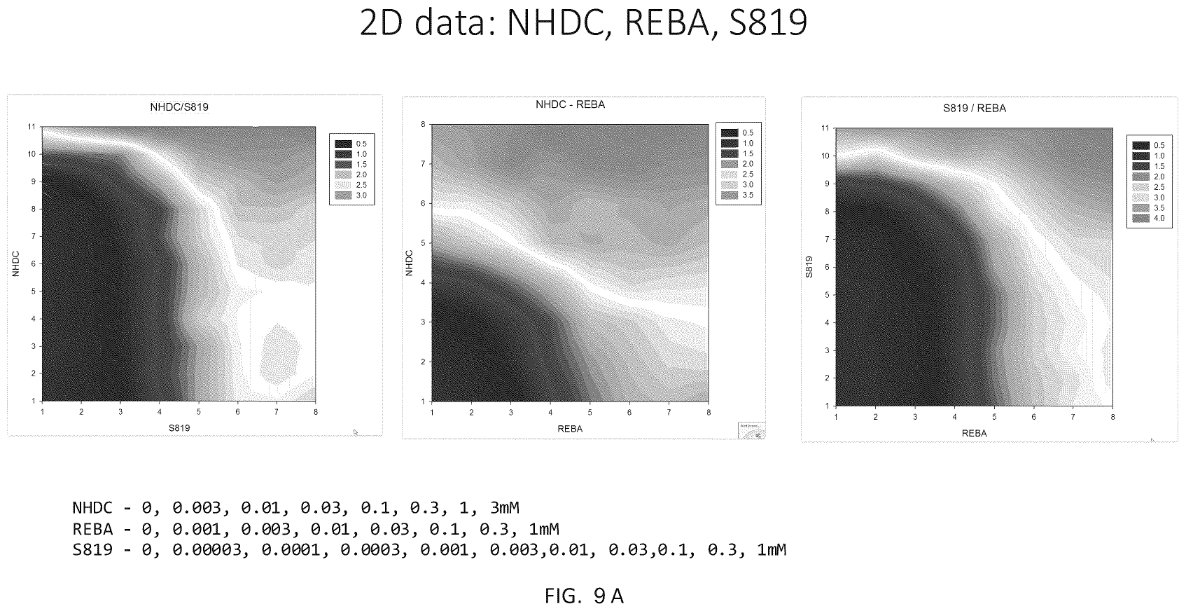

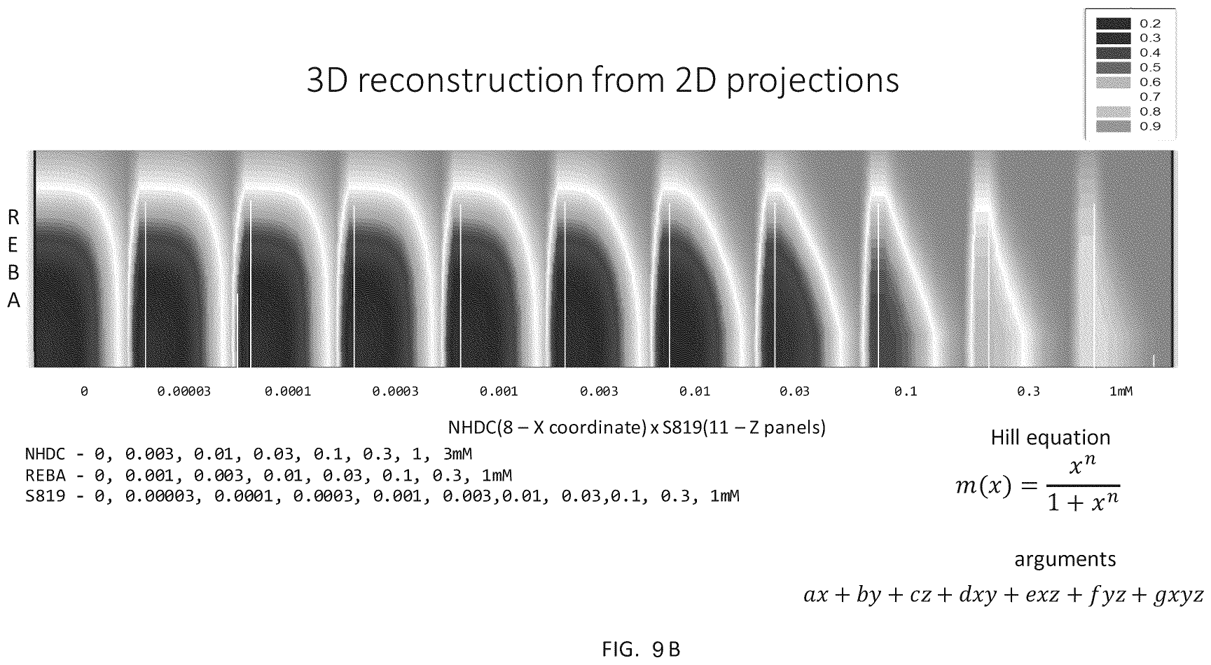

Methods: FIG. 9A shows the original 2D models of a mixture of NHDC and S819, a mixture of NHDC and REB A, and a mixture of S819 and REB A, respectively, as well as the concentrations of each of the sweeteners. FIG. 9B shows a 3D model reconstructed from the 2D models in FIG. 9A. A model using the Hill equation and arguments listed in FIG. 9B was generated based on the initial data in FIG. 9A. The Hills sigmoid function fit gave the following parameters: a=5.36, b=9.55, c=6.38, d=39.43, e=107.35, f=−79.31, g=81206, h=0.57.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method of generating a model configured to provide data for determining synergy between a plurality of compounds in a mixture for applying to a human taste receptor, the method comprising:
receiving, by a processor, a first data set comprising a plurality of data points, each data point comprising a concentration level of each compound in a mixture of a plurality of compounds;
receiving, by the processor, a second data set comprising a response level of a human taste receptor determined for each data point from the plurality of data points; and
generating, by the processor, a model of response levels of the human taste receptor based on the first data set and the second data set by:
determining, by the processor, a mapping between each data point from the plurality of data points and the response level of the human taste receptor determined for each data point from the plurality of data points;
determining, by the processor, a first function of the concentration level of each compound in the mixture, the function having one or more unknown coefficients; and
determining, by the processor, a second function of the concentration level of each compound in the mixture, the second function having the same form as the first function, by determining the one or more unknown coefficients by fitting the first function to the mapping between each data point from the plurality of data points and the response level of the human taste receptor determined for each data point from the plurality of data points.

2. The method of claim 1, the method further comprising: displaying data associated with the model to a user on a user device.

3. The method of claim 1, wherein the human taste receptor is a sweet taste receptor T1R2/T1R3 and wherein each compound in the mixture is a sweet-tasting compound.

4. The method of claim 1, wherein the first function is a sigmoid function.

5. The method of claim 4, wherein the sigmoid function is based on a logistic function, a trigonometric function, or a Hill equation.

6. The method of claim 1, wherein each compound in the mixture is selected from the group consisting of sugars, mogrosides, sweet amino acids, polyols, artificial sweeteners, natural sweeteners, and sweet tasting proteins.

7. The method of claim 1, wherein the mixture comprises a first sweet-tasting compound, a second sweet-tasting compound, and a third sweet-tasting compound.

8. The method of claim 7, wherein each of the first sweet-tasting compound, the second sweet-tasting compound, and the third sweet-tasting compound is selected from aspartame, sucrose, sucralose, rebaudioside A, rebaudioside D, rebaudioside M, thaumatin, neohespheridin, and S819[1-((1H-pyrrol-2-yl)methyl)-3-(4-isopropoxyphenyl)thiourea].

9. A computer-implemented method of preparing a synergistic sweetener composition using a sweet taste receptor response model, the method comprising:
receiving, by a processor, a plurality of data points associated with a plurality of sweeteners, each data point indicating a concentration level of a corresponding sweetener among the plurality of sweeteners;
determining, by the processor and using the sweet taste receptor response model, synergy among the plurality of sweeteners based on the received plurality of data points, wherein the synergy is measured by an increase in a response level of a sweet taste receptor to the plurality of sweeteners; and
prompting, by the processor and via a user interface, a user to prepare a synergistic sweetener composition including the pluraity of sweeteners in the respective concentration levels indicated in the plurailty of data points, such that the synergistic sweetener composition produces the synergy determined using the sweet taste receptor response model.

10. The method of claim 9, wherein the increase in the response level of the sweet taste receptor is equal to or greater than 25%.

11. The method of claim 9, wherein the plurality of sweeteners comprise at least two sweeteners.

12. The method of claim 9, wherein the plurality of sweeteners comprise at least three sweeteners.

13. The method of claim 9, further comprising:
displaying, by the processor and via the user interface, the response level of the sweet taste receptor.

14. The method of claim 13, wherein the response level of the sweet taste receptor is displayed as luminosity.

15. The method of claim 14, wherein the response level of the sweet taste receptor is displayed in a graph.

16. The method of claim 9, wherein the sweet taste receptor response model is trained by:
determining, by the processor, a mapping between sample data points of sample sweeteners and corresponding response levels of the sweet taste receptor, each sample data point including a concentration level of a corresponding sample sweetener;
determining, by the processor, a first function of the concentration levels of the sample sweeteners, the function having one or more unknown coefficients; and
determining, by the processor, a second function of the concentration levels of the sample sweeteners, the second function having the same form as the first function, by determining the one or more unknown coefficients by fitting the first function to the mapping between the sample data points and the corresponding response levels of the sweet taste receptor.

17. A method of preparing a synergistic sweetener composition based on a sweet taste receptor response model, the method comprising:
preparing a synergistic sweetener composition with two or more sweeteners in respective concentration levels recommended by a sweet taste receptor model;
wherein the model is generated based on a plurality of data points associated with sweetener mixtures, each data point comprising a concentration level of each sweetener in each sweetener mixture and sweet taste receptor data comprising each response level of a sweet taste receptor T1R2/T1R3 determined for each data point from the plurality of data points; and
wherein the model is analyzed to determine synergy among sweeteners in sweetener mixtures and respective concentration levels of the sweeteners at which synergy is demonstrated in order to provide a recommendation for a synergistic composition.

18. A sweetener composition prepared according to the method of claim 17.

19. A confectionery product comprising the sweetener composition of claim 18.

20. The confectionery product of claim 19, wherein the confectionery product is selected from a group consisting of hard candy, soft candy, mints, chewing gum, gelatins, chocolate, fudge, jellybeans, fondant, licorice, hard-panned candy, toffee, and taffy.
